(19) United States
(12) Patent Application Publication
(10) Pub. No.: US 2023/0165897 A1
(43) Pub. Date: Jun. 1, 2023

(54) A PREPARATION FOR PERIODONTAL TISSUE AND A KIT COMPRISING THE SAME

(71) Applicant: Mari SAKAI, Tokyo (JP)

(72) Inventor: Mari SAKAI, Tokyo (JP)

(21) Appl. No.: 17/922,594

(22) PCT Filed: Apr. 6, 2022
(86) PCT No.: PCT/JP2022/017134

§ 371 Date: Oct. 31, 2022

(30) Foreign Application Priority Data
Apr. 12, 2021 (JP) 2021-067160

(51) Int. Cl. A61K 35/16; A61K 35/28; A61K 35/35; A61K 35/36; A61K 47/36; A61P 1/02; A61K 9/00

(52) U.S. Cl. CPC A61K 35/16; A61K 35/28; A61K 35/35; A61K 35/36; A61K 47/36; A61P 1/02; A61K 9/0019

(57) ABSTRACT

[PROBLEMS] The purpose of the present invention is to provide a novel preparation for a periodontal tissue and a kit including the preparation that can be used for treatment by a simple operation and has high safety and high periodontal tissue regeneration performance.

[SOLUTION] The present invention relates to a preparation for a periodontal tissue, comprising autologous conditioned serum and hyaluronic acid. In particular, the preparation for a periodontal tissue of the present invention, wherein the culture supernatant is obtained by a method comprising: a first culturing step of culturing cells by using a first medium; a second culturing step of culturing, after the first culturing step, the cells by using, as a culture medium, a second medium different from the first medium; and a step of obtaining, after the second culturing step, a culture supernatant containing the second medium. The second medium can comprise a calcium ion and a buffering agent, wherein the buffering agent can be selected from a Good's buffer, in particular HEPES.

### TECHNICAL FIELD

The present invention relates to an autologous conditioned serum-containing preparation for a periodontal tissue, a kit comprising the preparation, and a surgical method using the preparation.

### BACKGROUND ART

Periodontal tissues are damaged by periodontal disease. As the periodontal disease progresses, what is called a periodontal pocket occurs between the tooth and the gum, resulting in gingival recession. Further periodontal disease progress causes alveolar osteolysis and tooth mobility. Eventually, the tooth should be extracted. Periodontal tissues may also be damaged by orthodontics.

Various methods have been conventionally studied for regenerating a periodontal tissue affected by, for instance, periodontal disease. For example, bone grafting, GTR method, and periodontal tissue regeneration therapy using enamel matrix protein have been conventionally conducted.

In recent years, a periodontal tissue regenerator called Regroth (trademark) has been marketed. Regroth contains a protein called fibroblast growth factor as a main component, and is said to be capable of promoting the proliferation and differentiation of bone, muscle, adipocytes, etc. In addition, since this has a potent angiogenic effect, the periodontal tissue is believed to be able to be regenerated. However, the procedure requires periodontal surgery and is very expensive.

For gingival regeneration, hyaluronic acid is also injected. The injection of hyaluronic acid is effective for the gingival regeneration if the injection is performed by an appropriate method, but depending on the physical conditions and/or physical characteristics of a patient, the effect may be difficult to appear. The injection was ineffective for alveolar bone regeneration.

Conventionally, autologous conditioned serum has also been used for treatment of osteoarthritis (Patent Document 1). Such autologous conditioned serum has recently attracted attention in the field of anti-aging (Patent Document 2).

### PRIOR ART DOCUMENTS

### Patent Documents

Patent Document 1: JP2002-540818A
Patent Document 2: JP2019-528281A

### SUMMARY OF THE INVENTION

### Problems to be Solved by the Invention

The purpose of the present invention is to provide a novel preparation for a periodontal tissue and a kit including the preparation that can be used for treatment by a simple operation and has high safety and high periodontal tissue regeneration performance.

### Means for Solving the Problems

The inventors discovered that the above problem can be solved by the present invention having the following aspects.

<<Aspect 1>>

A preparation for a periodontal tissue, comprising autologous conditioned serum.

<<Aspect 2>>

The preparation for a periodontal tissue according to aspect 1, further comprising a culture supernatant.

<<Aspect 3>>

The preparation for a periodontal tissue according to aspect 2, wherein the culture supernatant is obtained by a method comprising:

a first culturing step of culturing cells by using a first medium;

a second culturing step of culturing, after the first culturing step, the cells by using, as a culture medium, a second medium different from the first medium; and

a step of obtaining, after the second culturing step, a culture supernatant containing the second medium.

<<Aspect 4>>

The preparation for a periodontal tissue according to aspect 3, wherein the second culturing step is performed in a non-CO₂ atmosphere.

<<Aspect 5>>

The preparation for a periodontal tissue according to aspect 3 or 4, wherein the second medium comprises a calcium ion and a buffering agent.

<<Aspect 6>>

The preparation for a periodontal tissue according to aspect 5, wherein the buffering agent is selected from a Good's buffer.

<<Aspect 7>>

The preparation for a periodontal tissue according to aspect 6, wherein the Good's buffer is HEPES.

<<Aspect 8>>

A kit for a periodontal tissue, comprising the preparation for a periodontal tissue according to any one of aspects 2 to 7 and a hyaluronic acid-containing preparation for a periodontal tissue.

<<Aspect 9>>

The preparation for a periodontal tissue according to any one of aspects 1 to 8, wherein the autologous conditioned serum is obtained by a method comprising the steps of:

incubating blood collected from a patient using the preparation for a periodontal tissue to enrich cytokines and/or growth factors in the blood; and

separating serum from the cytokines- and/or growth factors-enriched blood.

<<Aspect 10>>

The preparation for a periodontal tissue according to aspect 9, wherein the step of enriching the cytokines and/or the growth factors is performed by bringing the blood into contact with glass.

<<Aspect 11>>

A method for producing an autologous conditioned serum- and culture supernatant-containing preparation for a periodontal tissue, wherein the culture supernatant is obtained by a method comprising:

a first culturing step of culturing cells by using a first medium;

a second culturing step of culturing, after the first culturing step, the cells by using, as a culture medium, a second medium different from the first medium; and

a step of obtaining, after the second culturing step, a culture supernatant containing the second medium, and wherein

the autologous conditioned serum is obtained by a method comprising the steps of:

incubating blood collected from a patient using the preparation for a periodontal tissue to enrich cytokines and/or growth factors in the blood; and

separating serum from the cytokines- and/or growth factors-enriched blood.

### BRIEF DESCRIPTION OF THE DRAWINGS

FIG. 1a shows panoramic X-ray photographs of a patient with alveolar bone defects caused by periodontal disease and bruxism.

FIG. 1B shows panoramic X-ray photographs of the patient after multiple injections of a preparation of the invention into the patient of FIG. 1a. The alveolar bone of the maxillary anterior tooth in a portion indicated by the arrow increased, and the slight mobility (M1) before surgery was also improved.

FIG. 2a shows a panoramic X-ray photograph of a patient who had a strong hit on the maxillary anterior teeth part and had slight mobility (grade II) and palpation pain in the right and left anterior teeth.

FIG. 2b shows a panoramic X-ray photograph of the patient after multiple injections of a preparation of the invention into the patient of FIG. 2a. It can be seen that the bone density of the maxillary anterior tooth in the portion indicated by each arrow increases and the position of the alveolar crest becomes higher.

FIG. 3a shows panoramic X-ray photographs of a patient with bone resorption and loss observed at the mandibular left molar (mandibular left at tooth position 7) on the mesial side due to periodontal disease deterioration caused by smoking.

FIG. 3b shows panoramic X-ray photographs of the patient after multiple injections of a preparation of the invention into the patient of FIG. 3a. In the portion indicated by the arrow, new bone starts to be formed, the white area increases, and an alveolar bone line is formed.

FIG. 4a shows a state of a periodontal tissue of a patient with gingival recession. The gum of the maxillary anterior teeth and the gums of the mandibular anterior teeth as indicated by the arrows regress, resulting in large black triangles.

FIG. 4b shows a state of the gums of the patient after multiple injections of a preparation of Example 2 into the gums of the patient of FIG. 4a. The gums of the maxillary gingiva and the mandibular gingiva as indicated by the arrows recovered, and the black triangles disappeared or improved.

FIG. 5a shows a front photographic view of a periodontal tissue of a patient with gingival recession. The gums of the maxillary anterior teeth and the gums of the mandibular anterior teeth as indicated by the arrows regress, resulting in black triangles.

FIG. 5b shows a state of the gums of the patient after multiple injections of a preparation of Example 2 into the gums of the patient of FIG. 5a. The gums of the maxillary gingiva and the mandibular gingiva as indicated by the arrows recovered, and the black triangles disappeared or improved.

FIG. 6a is a photograph of the patient of FIG. 5a as taken from the right side.

FIG. 6b is a photograph of the patient of FIG. 5b as taken from the right side.

FIG. 7a is a photograph of the patient of FIG. 5a as taken from the left side.

FIG. 7b is a photograph of the patient of FIG. 5b as taken from the left side.

FIG. 8a shows a state of a periodontal tissue of a patient with gingival recession. The gum of the maxillary anterior tooth and the gum of the mandibular anterior tooth as indicated by the arrows regress, resulting in black triangles.

FIG. 8b shows a state of the gums of the patient after multiple injections of a hyaluronic acid-containing preparation into the gums of the patient of FIG. 48. The gums of the maxillary gingiva and the mandibular gingiva as indicated by the arrows recovered, and the black triangles disappeared or improved.

FIG. 9a shows a state of a periodontal tissue of a patient with gingival recession.

FIG. 9b shows a state of the alveolar bones of the patient after multiple injections of a preparation just containing a culture supernatant into the gums of the patient of FIG. 59.

FIG. 10a shows a state of a periodontal tissue of a patient with alveolar bone defects caused by severe periodontal disease. The left arrow in the photograph indicates the alveolar bone in which the defects are observed. The right arrow in the photograph indicates the periapical bone of the canine.

FIG. 10b shows a state of the alveolar bones of the patient after multiple injections of a preparation just containing a culture supernatant into the alveolar bones of the patient of FIG. 10a. The left arrow in the photograph indicates that the alveolar bone is regenerated at the missing teeth portion. The right arrow in the photograph indicates that the periapical bone of the canine is being regenerated.

FIG. 11a shows a state of a periodontal tissue of a patient with a periodontal tissue destroyed due to severe periodontal disease. The arrow in the left photograph of FIG. 11a indicates recession caused by the alveolar bone defects. The arrow in the right photograph of FIG. 11a indicates the thinned periapical bone of the canine.

FIG. 11b shows a state of the alveolar bones of the patient after multiple injections of a preparation just containing a culture supernatant into the gums of the patient of FIG. 11a. The arrow in the left photograph of FIG. 11b indicates that the alveolar bone is regenerated, resulting in reduced recession. The arrow in the right photograph of FIG. 11b indicates that the periapical bone of the canine is being regenerated.

### DETAILED DESCRIPTION OF THE INVENTION

The preparation for a periodontal tissue according to the present invention contains autologous conditioned serum as an active ingredient. The preparation for a periodontal tissue means, in one example, a preparation for regenerating a periodontal tissue.

The periodontal tissue herein includes a tooth and tissues around the tooth. Examples includes crown portions (e.g., enamel, dentin, dental pulp, gum) and tooth root portions (e.g., cement, alveolar bone, blood vessel, nerve). The preparation for a periodontal tissue according to the present invention relates to a preparation for regenerating, in particular, the gum of crown portion and the alveolar bone of tooth root portion. In one example, the preparation for a periodontal tissue may be a preparation for treating periodontal disease or alveolar pyorrhea.

The present inventors have found that for example, a periodontal disease-affected periodontal tissue can be regenerated very effectively and easily by an autologous conditioned serum-containing preparation. Specifically, the conventional regeneration protocol has required periodontal surgery. Its effects may vary and its safety may be low depending on the practitioner. These are problems. By contrast, according to the preparation of the present invention, the preparation for a periodontal tissue should be just injected at or near a periodontal tissue to be regenerated. The regenerative effects have also been found to be markedly high. In particular, the alveolar bone has not been substantially regenerated by the conventional regeneration protocol. By contrast, the preparation of the present invention has been found to make it possible to very effectively regenerate an alveolar bone. This effect has been found to be remarkable when both autologous conditioned serum and a culture supernatant are included.

<Autologous Conditioned Serum>

The autologous conditioned serum is serum obtained by adjusting blood collected from a patient himself/herself who uses the preparation and separating the blood, and provided such that the levels of cytokines and/or growth factors are much higher than in normal blood. For example, the levels of cytokines and/or growth factors contained in the autologous conditioned serum is twice or more or three times or more the levels in normal blood, and may be about five times the levels. The autologous conditioned serum is highly safe because it is made of blood collected from a patient himself/herself. Also, the levels of cytokines and/or growth factors are high. Therefore, it has been found to be very useful for regeneration of a periodontal tissue, particularly regeneration of alveolar bone.

The autologous conditioned serum may be produced by a method comprising the steps of: incubating blood collected from a patient to enrich cytokines and/or growth factors in the blood; and separating serum from the cytokines- and/or growth factors-enriched blood. As such autologous conditioned serum, the autologous conditioned serum as described in Patent Documents 1 and 2 can be used. In addition, a kit for preparing autologous conditioned serum is commercially available, and examples of such a product include Sanakin (trademark). Examples of each cytokine include IL-1Ra, IL-4, IL-6, IL-10, or TNF-α. Examples of each growth factor include epidermal growth factor (EGF) or fibroblast growth factor (FGF). In the autologous conditioned serum, at least one of these cytokines may be enriched by 2-fold or more, 3-fold or more, or 5-fold or more as compared to normal serum.

The step of incubating blood collected from a patient using a preparation for a periodontal tissue to enrich cytokines and/or growth factors in the blood may be divided into a step of collecting blood from a patient and a step of incubating the collected blood to enrich cytokines and/or growth factors in the blood. The step of collecting blood from a patient is a step of collecting blood of a patient himself/herself using a preparation for a periodontal tissue, and for example, about 10 ml of blood can be collected with a common syringe.

In the step of incubating the collected blood to enrich cytokines and/or growth factors in the blood, the incubation may be performed, for example, in a range of 30° C. to 45° C., 35° C. to 40° C., or 36° C. to 38° C. for 30 minutes or longer, 1 hour or longer, or 2 hours or longer. The incubation time may be 1 day or less, half a day or less, or 5 hours or less. Further, this step may follow a step of transferring the collected blood to a container.

When the blood is incubated, the blood is preferably brought into contact with a solid surface such as glass or polymer (particularly, glass). In this way, the production of cytokines and/or growth factors can be promoted. To achieve this, it is preferable to increase the surface in contact with blood by putting, into a container for storing blood, not only the blood but also particles such as glass beads and/or solid material such as fibers (e.g., glass wool).

The container for storing blood may be a syringe that has been used for collecting blood, or may be a container different from the syringe. The material constituting the solid particles to be put into the container may be a material different from the material constituting the container body. For example, the container can be made of a polymer and the solid material may be, for example, glass beads. For example, the diameter of the particles may range from 0.5 to 10 mm or 1 to 5 mm. According to a preferred embodiment, the solid material contained in the syringe may be less than 70% or less than 50 vol % of the internal volume of the syringe. The container may have a volume of, for instance, 10 to 100 ml.

In a particularly advantageous configuration of the invention, the container and the solid material are made of glass, quartz glass, corundum, quartz, polystyrene, polyethylene, polyvinyl chloride, plastic such as polypropylene, etc. Alternatively, these substances or a mixture thereof may be substantially included.

In particular, the solid surface in contact with the blood may be glass. In this case, the effect of producing cytokines and/or growth factors is very strong. In addition, an IL-6 receptor antibody, which exerts a very potent effect of regenerating a periodontal tissue, has been found to be produced particularly in a large amount. Thus, the autologous conditioned serum as so produced has been found to exert a remarkable effect when used as a preparation for a periodontal tissue.

Centrifugation may be used to perform a step of separating serum from the cytokines- and/or growth factors-enriched blood. The centrifugation may be performed at a normal speed used to obtain serum, and is performed at, for instance, 500 to 2000 g for several minutes. For example, when 10 ml of blood is collected, about 3 to 5 ml of the cytokines- and/or growth factors-enriched serum can be collected after centrifugation. Platelets and blood cell components should be removed with a cell filtration filter from the collected serum so as not to contain them.

### Culture Supernatant

In the present specification, a culture supernatant refers to a culture liquid obtained by culturing cells, which does not substantially contain a cell. The mesenchymal cells or the stem cells used for obtaining a culture supernatant may be derived from human, a non-human mammal, an insect, a bird, or a plant. In order to prevent stimulating an immune reaction, the cells are preferably derived from human, in particular, from cells or stem cells of mesenchymal or epidermis. More preferably, the cells are adipose tissue-derived mesenchymal stromal cells, cord blood stem cells, epidermis-derived epithelial cells, or dental pulp-derived mesenchymal stem cells. In particular, the cells are adipose tissue-derived mesenchymal stromal cells or cord blood stem cells.

The culturing method may appropriately be adjusted depending on cells to be cultured. Culturing can be performed under any condition suitable for culturing mammalian cells, if mammalian cells are used. In general, cells are cultured for several days at 37° C. and 5% CO2, and the medium is replaced as needed.

Although the types of mediums are not limited, an example of the medium includes α-MEM or DMEM supplemented with 10 to 15% autoserum or fetal bovine serum (FBS) and antibiotics. A medium free from a human- or animal-derived component may also be used. A growth factor, such as fibroblast growth factor (bFGF) or adrenomedullin, may be added as needed. Further, the mediums listed below can be used for the second medium.

The culture supernatant may be produced by the method comprising the first culturing step of culturing cells using the first medium, the second culturing step of culturing cells using the second medium that is different from the first medium after the first culturing step, and the step of obtaining the culture supernatant containing the second medium after the second culturing step.

In the first culturing step, a known culturing method is appropriately adopted. The first culturing step is a step for growing cells to a confluent state or a sub-confluent state using a first medium.

Any mediums can be used for the first medium, if it can grow cells to be used. An example of the first medium includes α-MEM or DMEM supplemented with 10 to 15% autoserum or fetal bovine serum (FBS) and antibiotics. A medium free from a human- or animal-derived component may also be used. A growth factor, such as fibroblast growth factor (bFGF) or adrenomedullin, may be added as needed.

After the first culturing step, the cells can be recovered and rinsed. The recovered cells can be cultured using the second medium that is different from the first medium.

In the second culturing step, it is preferable that a CO2 incubator is not used and CO2 culturing is not performed. In the preferably embodiment, CO2 culturing is not necessary for the culture supernatant. That is, culturing is preferably performed using a culture container without performing CO2 incubation at about 37° C. The second culturing step is preferably a step of culturing cells from 5 hours to 5 days (from 10 hours to 2 days or from 5 hours to 3 days). Culturing may be performed by adhesion culture or suspension culture depending on cells to be cultured. Ina case where the cells are removed, culturing may be performed by a method in which the cells are easily removed.

Although the mediums listed above for the first medium and other well-known mediums can be used for the second medium, serum-free media can be preferably used in order to prevent stimulating an immune reaction. The medium prepared by adding a calcium ion and a buffering agent can be used as the second medium. The second medium is preferably an electrolytic solution not only contains a calcium ion and a buffering agent, but also optional prostaglandin. If it further contains prostaglandin, it is found that the advantageous effect can be obtained. All water-soluble and biotolerant calcium salts, such as calcium chloride, can be the source of the calcium ion.

The second medium may comprise an infusion liquid for injection or an infusion liquid for intravenous drip infusion. In this case, a product manufactured and sold as an infusion liquid for injection or an infusion liquid for intravenous drip infusion can be appropriately used. Examples of the infusion liquid for injection include a sugar liquid agent, an extracellular fluid replacement fluid (a physiological saline solution, a Ringer's solution, a Ringer's lactate solution, an extracellular fluid replacement fluid, a Ringer's acetate solution, a Ringer's bicarbonate solution), a hypotonic electrolyte fluid, an amino-acid preparation (a high-concentration amino acid solution, an amino acid solution for renal failure, an amino acid solution for liver failure, an amino acid solution for child), peripheral parenteral nutrition (PPN), total parenteral nutrition (TPN), fat emulsion, and a plasma volume expander. Of these, an extracellular fluid replacement fluid (a physiological saline solution, a Ringer's solution, a Ringer's lactate solution, an extracellular fluid replacement fluid, a Ringer's acetate solution, a Ringer's bicarbonate solution) and an isotonic electrolyte fluid are preferable. Specific examples of the infusion liquid include PAREPLUS®.

If the second medium contains calcium ions, the calcium ion is preferably 0.045 mM to 1.802 mM. It may be 0.074 mM to 1.505 mM, 0.045 mM to 2 mM, 0.180 mM to 2 mM, 1 mM to 2 mM, 1.3 mM to 1.8 mM, 1.2 mM to 1.6 mM, 1 mM to 1.6 mM, 0.045 mM to 1.352 mM, 0.180 mM to 0.901 mM, or 20 mg/l to 100 mg/l. Examples of a salt included in the second medium include NaCl, KCl, and CaCl2, and the salt may be included in an amount of 1 g/L to 30 g/L, 4 g/L to 30 g/L, or 6 g/L to 11 g/L.

Examples of the buffering agent include inorganic buffering agents, such as MgSO4.7H2O, Na2HPO4, KH2PO4, NHHCO3 and organic buffering agent, such as Goods buffers. Examples of Goods buffers include MES, bis-trismethane, ADA, PIPES, ACES, MOPSO, colamin hydrochloride, MOPS, BES, TES, HEPES, DIPSO, MOBS, acetamidoglycine, TAPSO, TEA, POPSO, HEPPSO, EPS, HEPPS, tricine, Tris, glycinamide, glycylglycine, HEPBS, bicine, TAPS, AMPB, CHES, AMP, AMPSO, CAPSO, CAPS, CABS. Among these, HEPES (hydroxyethylpiperazine ethane sulfonic acid) can be preferably used. These may be used in combination with other salts (e.g., sodium hydrogen carbonate, sodium carbonate, pyruvic acid, citric acid, salts thereof, and the like). The content of these buffering agents can be selected depending on the PH range to be controlled, and is preferably 1 mg/l to 5 g/l, and it may be 2 mg/l to 500 mg/l or 10 mg/l to 300 mg/l.

The acidity of the second medium is, for example, pH5.5 to pH9, and it may also be pH7.2 to pH7.8.

The second medium preferably includes less sugar, and the content of the sugar (e.g., glucose) is preferably 1 g/l or less. It may be 0.8 g/l or less, 0.5 g/l or less, 0.1 g/L to 1.5 g/L, 0.1 g/L to 1.2 g/L, 0.1 g/L to 1 g/L, 0.5 g/L to 1.2 g/L, or 0.8 g/L to 1.1 g/L.

Further, the second medium preferably includes less, substantially no, or no amino acids, and the amino acid content is preferably 1 mg/ml or less, preferably 0.8 mg/l or less, further preferably 0.5 mg/l or less.

The second medium preferably includes less, substantially no, or no vitamins, and the content of the vitamins is preferably 1 mg/ml or less, preferably 0.8 mg/l or less, further preferably 0.5 mg/l or less. The second medium is preferably free from an antibiotic (e.g., penicillin), a growth factor, and a cytokine.

The second medium preferably includes less or no heavy metal elements such as iron, copper, and lead and trace elements. Having the small amounts of such elements makes it possible to prevent the synthesis of metalloproteins and promote the synthesis of growth factors. The second medium is preferably free from a carcinogenic substance such as a polyamine (e.g., Putrescine 2HCl). The second medium is preferably free from a purine base. Having no purine base in the second medium makes it possible to activate a salvage pathway of nucleic acids.

Having no or small amounts of vitamins and amino acids in the second medium makes it possible to increase the synthesis of growth factors by promoting autophagy. The second medium can maintain a stable environment during culturing in a general culturing environment (e.g., the second medium can reduce fluctuation of the acidity and maintains the buffering capacity during culturing, making it unnecessary to perform CO2 culturing or the like).

The second medium preferably has the higher water content as compared with a normal medium. For example, the water content is preferably 95 wt. % to 99.99 wt. %, when the second medium is taken as 100 wt. %. It may be 96 wt. % to 99.9 wt. % or 97 wt. % to 99.9 wt. %. Having the higher water content in the medium can reduce the osmotic pressure. For example, when cells are grown by adherent culture, the cells are usually detached from a culture container by using an animal-derived digestive enzyme such as trypsin. Using the second medium eliminates the necessity of using a digestive enzyme. This can reduce side effects such as infection caused by an animal-derived component when an agent including the second medium is administered to a patient.

The second medium may be composed of only sugar (e.g., glucose), salts (salts including a calcium ion source, e.g., the ones composed of only NaCl, KCl, and CaCl2, or salts including these as main salts), and buffering agents (e.g., buffering agents composed of only MgSO4.H2O, Na2HPO4.2H2O, KH2PO4, NaHCO₃, and Good buffers, especially, HEPES, or buffering agents including these as main buffering agents) with the remainder being a solvent (e.g., water). The second medium having such a composition causes the high expression of specific genes or proteins and functions as a medium and an infusion liquid having excellent biocompatibility, as the effectiveness of the second medium is confirmed by Examples below.

The second medium having the low amino acid content avoids an interaction (mainly oxidation and reduction) and a polymerization reaction with synthesized and secreted growth factors, thereby making it possible to prevent deterioration of the components and also moisture absorption and deterioration caused by the contaminated amino acids. Further, the low amino acid content in the second medium reduces the risk of bacteria growth when the second medium is used as a raw material for cosmetics, making it possible to ensure the preservability of products without adding a preservative or an antioxidant. The second medium free from amino acids does not require a step of removing amino acids when applied to an affected site, and the second medium does not serve as nutrients for growing bacteria or the like residing in an affected site. This means that the second medium does not cause malodor when applied to a wound surface. Further, after the second medium is formulated, an agent including the second medium does not cause dysgeusia or body odor when applied by injection, etc, thus the second medium is highly convenient when it is used as a medicine. Desalting can be easily performed with the second medium free from amino acids. This makes it easy to obtain a high-concentration dried growth factor product by freeze drying when cells are grown in mass culture. When a normal medium is used, the medium needs to be exchanged after 48 to 72 hours under any circumstance. In a case where the second medium is not an amino acid-containing medium, the living environment can be maintained for a long period of time (e.g., about 7 days) by suppressing metabolic activity of the cultured cells stored in a cold place at 4° C. Subsequently, the cells can be propagated again by exchanging the medium. The second medium preferably including a calcium ion and a buffering agent preferably has a simple composition. Thus, the second medium having high versatility can be applied to any types of animal cells (including ES cells, iPS cells, and stem cells) and plant cells (in particular, callus culture of plants and maintenance of plant stem cells).

The step of obtaining the culture supernatant containing the second medium is carried out after the second culturing step. This step preferably further comprising a step of adding trehalose. The culture supernatant preparation preferably includes the second medium after the second culturing step in an amount of 50 wt. % to 100 wt. %. The culture supernatant preparation may include the second medium (including the culture supernatant) after the second culturing step in an amount of 60 wt. % to 100 wt. %, preferably in an amount of 70 wt. % to 99 wt. %, 70 wt. % to 90 wt. %, 80 wt. % to 99 wt. % 90 wt. % to 100 wt. % or 90 wt. % to 95 wt. % As the culture supernatant of stem cells or the like, a supernatant component, which is obtained by subjecting the culture supernatant to solid-liquid separation by centrifugal separation, is usually used. In the method described in this specification, the second medium can be actively included in a preparation, allowing a filtrate simply obtained by filtering the medium after the second culturing step to be used.

The preparation may be a treated material obtained by removing the water content by freeze drying from the culture supernatant obtained as described above, a treated material obtained by concentrating the culture supernatant under a reduced pressure using an evaporator or the like, a treated material obtained by concentrating the culture supernatant using an ultrafiltration membrane or the like, a treated material obtained by subjecting the culture supernatant to solid-liquid separation using a filter, or the original liquid of the culture supernatant not subjected to the above treatments. Further, for example, the sterile culture supernatant may be obtained by subjecting the supernatant of the cell culture to centrifugal separation (e.g., 1,000×g, 10 minutes), fractionating the supernatant using ammonium sulfate (e.g., saturated 65% ammonium sulfate), suspending a precipitate using an appropriate buffer solution, subjecting the resulting product to a dialysis treatment, and filtering the resulting dialysate using a syringe filter (e.g., 0.2 μm). The culture supernatant thus collected can be used as it is, or it can be frozen, stored and thawed when used. Further, the culture supernatant may be added with a pharmaceutically acceptable carrier and aliquoted into sterilized containers in a liquid amount of, for example, 0.2 ml or 0.5 ml for easy handling. Further, the culture supernatant may be treated with a virus clearance filter or γ-ray irradiation as a countermeasure against risks of infectious pathogens.

As described above, the freezing step of freezing the culture supernatant recovered in the culture supernatant recovery step may be included after the second culturing step and the culture supernatant recovery step of recovering the culture supernatant. For freezing the culture supernatant, for example, the culture supernatant is cooled from −200° C. to 0° C., and it may be cooled from −100° C. to −5° C. Note that the culture supernatant preparation may be obtained by disrupting the cells after the second culturing step, performing centrifugal separation, and then filtering the supernatant using a filter, or it may be obtained by further freezing and drying the filtrate thus obtained.

The preparation for periodontal tissue of the present invention may be produced by a known method, and may be administered by a known method, such as an injection, an application, to a periodontal tissue. As an example, the preparation can be administered to a gingiva or a alveolar bone. The preparation can be administered to a single site or plural sites. The preparation of the present invention can be administered at the amount of 0.1 mL to 3.0 mL, preferably 0.2 mL to 2.0 mL, 0.3 mL to 1.5 mL, or 0.5 mL to 1.2 mL, as an administration unit.

The most preferable culture supernatant is a culture supernatant disclosed in WO2020/027336A.

<Hyaluronic Acid>

The preparation for a periodontal tissue according to the present invention may be included in a kit for a periodontal tissue, further comprising a hyaluronic acid-containing preparation for a periodontal tissue. In particular, the preparation for a periodontal tissue according to the present invention preferably contains autologous conditioned serum, a culture supernatant, and hyaluronic acid. As used herein, the term “hyaluronic acid” encompasses all variants and combinations of variants of hyaluronic acid, hyaluronate, or hyaluronan of various chain lengths and charge states as well as with various chemical modifications including crosslinking.

Autologous conditioned serum-containing preparations are very expensive, and may take time to produce an effect. Thus, it is assumed that a patient who will discontinue treatment before the effect appears may be present. However, when hyaluronic acid is used in combination with the preparation of the present invention, the effect can be actually felt at an early stage. Because the effect can be actually felt at an early stage, this point is very helpful to continue the treatment. Thus, the kit containing the preparation of the present invention is a preparation that sufficiently provides patients with satisfaction.

Without being bound by theory, when hyaluronic acid is used, the hyaluronic acid injection site swells. This can resolve a black triangle caused by gum thinning. The hyaluronic acid is absorbed over time, but the gum does not return to its original position after the absorption. This seems to be because administration of hyaluronic acid causes swelling of gum to impart stretch stimulation to cells of the gum, thereby enhancing fibroblast activity (e.g., collagen synthesis potential, proliferation capability) to promote gingival regeneration. However, continuous administration is required for such an effect to be exerted. On the other hand, the autologous conditioned serum and the optional culture supernatant do not have an immediate effect, but can act on the gum and alveolar bone to promote regeneration of the periodontal tissue. These tissue regeneration potentials should be effectively exerted around tissues stretched by hyaluronic acid. This may exert a synergistic effect.

Hyaluronic acid is a kind of glycosaminoglycan mainly composed of glucuronic acid and N-acetylglucosamine. As explained above, the term “hyaluronic acid” encompasses all variants and combinations of variants of hyaluronic acid, hyaluronate or hyaluronan, of various chain lengths and charge states, as well as with various chemical modifications, including crosslinking. That is, the term also encompasses the various hyaluronate salts of hyaluronic acid with various counter ions, such as sodium hyaluronate. Various modifications of the hyaluronic acid are also encompassed by the term, such as oxidation, e.g. oxidation of —CH2OH groups to —CHO and/or —COOH; periodate oxidation of vicinal hydroxyl groups, optionally followed by reduction, e.g. reduction of —CHO to —CH2OH or coupling with amines to form imines followed by reduction to secondary amines; sulphation; deamidation, optionally followed by deamination or amide formation with new acids; esterification; crosslinking; substitutions with various compounds, e.g. using a crosslinking agent or a carbodiimide assisted coupling; including coupling of different molecules, such as proteins, peptides and active drug components, to hyaluronic acid; and deacetylation. Other examples of modifications are isourea, hydrazide, bromocyan, monoepoxide and monosulfone couplings.

The hyaluronic acid can be obtained from various sources of animal and non-animal origin. Sources of non-animal origin include yeast and preferably bacteria. The molecular weight of a single hyaluronic acid molecule is typically in the range of 0.1-10 MDa, but other molecular weights are possible.

The hyaluronic acid can be obtained from various sources of animal and non-animal origin. Sources of non-animal origin include yeast and preferably bacteria. The molecular weight of a single hyaluronic acid molecule is typically in the range of 0.1-10 MDa, but other molecular weights are possible. In certain embodiments the concentration of said hyaluronic acid is in the range of 1 to 100 mg/ml. In some embodiments the concentration of said hyaluronic acid is in the range of 2 to 50 mg/ml. In specific embodiments the concentration of said hyaluronic acid is in the range of 5 to 30 mg/ml or in the range of 10 to 30 mg/ml. In certain embodiments, the hyaluronic acid is crosslinked. Crosslinked hyaluronic acid comprises crosslinks between the hyaluronic acid chains, which creates a continuous network of hyaluronic acid molecules which is held together by the covalent crosslinks, physical entangling of the hyaluronic acid chains and various interactions, such as electrostatic interactions, hydrogen bonding and van der Waals forces.

Crosslinking of the hyaluronic acid may be achieved by modification with a chemical crosslinking agent. The chemical crosslinking agent may for example selected from the group consisting of divinyl sulfone, multiepoxides and diepoxides. According to embodiments the chemical crosslinking agent is selected from the group consisting of 1,4-butanediol diglycidyl ether (BDDE), 1,2-ethanediol diglycidyl ether (EDDE) and diepoxyoctane. According to a preferred embodiment, the chemical crosslinking agent is 1,4-butanediol diglycidyl ether (BDDE).

The crosslinked hyaluronic acid product is preferably biocompatible. This implies that no, or only very mild, immune response occurs in the treated individual. That is, no or only very mild undesirable local or systemic effects occur in the treated individual.

The crosslinked hyaluronic acid product according to the invention may be a gel, or a hydrogel. That is, it can be regarded as a water-insoluble, but substantially dilute crosslinked system of hyaluronic acid molecules when subjected to a liquid, typically an aqueous liquid.

The gel contains mostly liquid by weight and can e.g. contain 90-99.9% water, but it behaves like a solid due to a three-dimensional crosslinked hyaluronic acid network within the liquid. Due to its significant liquid content, the gel is structurally flexible and similar to natural tissue, which makes it very useful as a scaffold in tissue engineering and for tissue augmentation.

As mentioned, crosslinking of hyaluronic acid to form the crosslinked hyaluronic acid gel may for example be achieved by modification with a chemical crosslinking agent, for example BDDE (1,4-butandioldiglycidylether). The hyaluronic acid concentration and the extent of crosslinking affects the mechanical properties, e.g. the elastic modulus G′, and stability properties of the gel.

Crosslinked hyaluronic acid gels are often characterized in terms of “degree of modification”. The degree of modification of hyaluronic acid gels generally range between 0.1 and 15 mole %. Advantageously, the degree of modification of hyaluronic acid gels is a degree of modification of 2 mole % or less, such as 1.5 mole % or less, such as 1.25 mole % or less, for example in the range of 0.1 to 2 mole %, such as in the range of 0.2 to 1.5 mole %, such as in the range of 0.3 to 1.25 mole %, as compared to more crosslinked hyaluronic acid gels. The degree of modification (mole %) describes the amount of crosslinking agent(s) that is bound to HA, i.e. molar amount of bound crosslinking agent(s) relative to the total molar amount of repeating HA disaccharide units. The degree of modification reflects to what degree the HA has been chemically modified by the crosslinking agent. Reaction conditions for crosslinking and suitable analytical techniques for determining the degree of modification are all well known to the person skilled in the art, who easily can adjust these and other relevant factors and thereby provide suitable conditions to obtain a degree of modification in the range of 0.1-2% and verify the resulting product characteristics with respect to the degree of modification. A BDDE (1,4-butandiol diglycidylether) crosslinked hyaluronic acid gel may for example be prepared according to the method described in Examples 1 and 2 of published international patent application WO 9704012.

In a preferred embodiment the hyaluronic acid of the composition is present in the form of a crosslinked hyaluronic acid gel crosslinked by a chemical crosslinking agent, wherein the concentration of said hyaluronic acid is in the range of 10 to 30 mg/ml and the degree of modification with said chemical crosslinking agent is in the range of 0.1 to 2 mole %.

Hyaluronic acid gels may also comprise a portion of hyaluronic acid which is not crosslinked, i.e not bound to the three-dimensional crosslinked hyaluronic acid network. However, it is preferred that at least 50% by weight, preferably at least 60% by weight, more preferably at least 70% by weight, and most preferably at least 80% by weight, of the hyaluronic acid in a gel composition form part of the crosslinked hyaluronic acid network.

The types of hyaluronic acid can be selected depending on the periodontal tissue site where the preparation of the present invention is used. For example, if the preparation is injected to gingiva, a hyaluronic acid gel containing relatively small and soft particles, such as Restylane Vital Skin Boosters®, can be used.

### Miscellaneous

If the preparation is used as a liquid agent, the liquid agent can be produced by a known method. For example, it can be produced by mixing an autologous conditioned serum and an optional culture supernatant powder obtained by freeze dry with a pharmaceutically acceptable solvent and filling the resulting mixture in a sterilized container for liquid agent. In addition, a hyaluronic acid can be added to the sterilized container. Examples of the pharmaceutically acceptable solvent include water for injection, distilled water, physiological saline, an electrolyte solution agent, or a liquid agent having a composition equivalent to that of a culture liquid, and a sterilized solvent is preferably used. Examples of the sterilized container for liquid agent include an ampoule, a vial, a syringe, and a bag. For those containers, a known container made of glass, plastic, or the like can be used. Specific examples of the plastic-made container include a container made by using a material such as polyvinyl chloride, polyethylene, polypropylene, or a copolymer of ethylene and vinyl acetate. Examples of a sterilizing method of these containers and solvents include a heating method (a flame method, a drying method, a high-temperature steam method, a free-flowing steam method, a boiling method, or the like), a filtration method, an irradiation method (a radiation method, an ultraviolet method, a high-frequency method, or the like), a gas method, and a liquid chemical method. These sterilizing methods can be appropriately selected and used by a person skilled in the art depending on the material of the container and the characteristics of the solvent.

In one embodiment, if the preparation and the kit comprising the same of the present invention contain a culture supernatant, although the culture supernatant does not usually contain substantial cells, it may contain cultured cells. If the preparation of the present invention contains cultured cells, they may be preferably autologous cells or allogeneic cells, more preferably autologous cells. In a case where cells are used for treatment as a liquid agent, an injection to a periodontal tissue can be most frequently used as a transplanting method. For example, in the case of using the injection to a periodontal tissue, the liquid agent is prepared preferably 1×10⁵ cells/mL to 5×10⁷ cells/mL, more preferably 1×10⁶ cells/mL to 1×10⁷ cells/mL. Further, a mesenchymal stem cell agent is prepared preferably 1×10⁵ cells to 1×10⁹ cells, more preferably 2×10⁷ cells to 2×10⁸ cells, as an administration unit of the injection for human. As for other administration routes, the preparation can be used within a range of the liquid quantity which can be transplanted to the tissue and the maximum number of cells which can be suspended in that liquid quantity.

The preparation and the kit comprising the same of the present invention may be prepared with a pharmaceutically acceptable carrier or medium. Examples of the pharmaceutically acceptable carrier or medium include a pharmaceutically acceptable substance such as a stabilizer, a solubilizer, a suspending agent, a buffering agent, a tonicity agent, an antioxidant, or a preservative. Examples of the tonicity agent include sodium chloride and glucose. Examples of the buffering agent include a citrate, an acetate, boric acid, and a phosphate. As an aqueous medium for suspending cells, for example, an aqueous solution for injection or the like in which the osmotic pressure and pH are adjusted to near the blood values and the salt concentration or the like is adjusted is appropriately used. For example, a Ringer's solution such as a Ringer's acetate solution or a sugar containing Ringer's acetate solution and other infusion liquids, physiological saline, a glucose solution, or the like can be used without being limited thereto. For example, in a case of using a Ringer's solution for infusion, a Ringer's solution may be added with an acceptable amount of dimethyl sulfoxide (DMSO) or human serum albumin (HSA). Examples of the antioxidant include ascorbic acid, sodium hydrogen sulfite, and sodium metabisulfite. Examples of the preservative include phenol, thimerosal, and benzalkonium chloride.

The preparation or the kit containing the preparation of the present invention has been found to be very effective for women and men in their twenties to fifties, and particularly effective for women in their twenties to forties. The effects on women in their twenties to forties were remarkable in the case of a preparation containing autologous conditioned serum and a culture supernatant or a kit containing the preparation.

Note that the present specification also discloses a method for treatment of a periodontal tissue of a subject (human or non-human mammal) by using the above-described autologous conditioned serum-containing preparation for a periodontal tissue or a kit containing the preparation. In the case of treatment using them, for instance, an injection may be used for administration to an affected site.

The present invention will be described more specifically in the following Examples, but the present invention is not limited thereto.

### EXAMPLES

<<Preparation of Autologous Conditioned Serum>>

In order to produce autologous conditioned serum for use in a preparation of the present invention, 10 ml of blood was collected, using a syringe, from each patient.

The collected blood was transferred from the syringe to a Sanakin (trademark) tube, and the tube was inverted for mixing 3 to 4 times, and then stored in a storage maintained at 37° C. for 3 hours.

Thereafter, the Sanakin (trademark) tube was transferred to a centrifuge, and centrifuged at 3000 rpm (1200 G) for 5 minutes, so that platelets and blood cell components floating in serum were sedimented.

The serum in the supernatant portion as separated by centrifugation was collected in about 3 to 5 ml with a luer-lock syringe having a cathelin needle attached, and then platelets and blood cell components that could be contained in a small amount were removed with a cell filtration filter to prepare autologous conditioned serum.

<<Preparation of Culture Supernatant>>

In addition, the culture supernatant used in the preparation of the present invention was produced in substantially the same manner as in the method described in WO 2020/027336 A1. Specifically, the following procedure was used for the production.

<First Culturing Step>

Adipose tissue-derived stromal cells obtained by subjecting normal human adipose tissue to enzymatic treatment were cultured in a non-coated culture flask (manufactured by FALCON) while using 20% FBS (fetal bovine serum)-containing DME medium (Dulbecco's modified Eagle's medium, manufactured by Gibco; high glucose) to obtain a primary culture. The primary culture cells immediately before the cells became confluent were recovered by enzyme treatment, and then seeded on a non-coated 12-well culture plate (manufactured by Sumitomo Bakelite Co., Ltd.) in the same culture medium, and cultured until the cells became confluent.

After the cells were found to be confluent, the culture medium was removed, and the cell surface was washed with PBS (Dulbecco's phosphate buffer solution, manufactured by DS Pharma Biomedical Co., Ltd.).

<Second Culturing Step>

The washed cells were cultured while the culture medium was replaced by HBSS (Hank's Balanced Salt Solution, manufactured by Sigma-Aldrich)-HEPES. Thereafter, 12 wells were divided into 4 groups of 3 wells, and the cells were cultured in the cultureware without using CO₂.

<<Recovery of Culture Supernatant>>

The culture supernatant in each well was recovered 48 hours after the replacement.

HBSS (Hank's balanced salt solution) is a solution obtained by mixing (1) a sodium hydrogen carbonate solution, which is an isotonic liquid, (2) a buffer containing NaCl, KCl, MgSO₄.7H₂O, Na₂HPO₄, glucose, and KH₂PO₄, and (3) a CaCl₂ solution.

HEPES is hydroxyethyl piperazine ethanesulfonic acid, and is a buffering agent or a pH modifier.

The amount of carbohydrate contained in the HBSS-HEPES medium (hanks-HEPES) was 1.0 g/L, the amount of NaCl was 8.0 g/L, the amount of KCl was 0.4 g/L, the amount of CaCl₂) was 0.14 g/L, the amount of MgSO₄.H₂O was 0.2 g/L, the amount of Na2HPO₄.2H₂O was 0.06 g/L, the amount of KH₂PO₄ was 0.06 g/L, and the amount of NaHCO₃ was 0.35 g/L. Here, 20 mM HEPES (pH 7.4) was used, and the total amount of amino acids (e.g., L-glutamic acid) was 0 g/L and the calcium ion was at 1.5 mM.

<<How to Prepare Preparation>>

Here, 2 ml of autologous conditioned serum was prepared as a preparation for injection in Example 1. In addition, a lyophilized culture supernatant produced from 1 ml of the culture supernatant was dissolved in 1 ml of physiological saline. This was mixed with 1 ml of the autologous conditioned serum to prepare a preparation for injection in Example 2. Then, the lyophilized culture supernatant produced from 1 ml of the culture supernatant was just dissolved in 1 ml of physiological saline to prepare a preparation for injection as a preparation in Comparative Example.

<<Pharmacological Effects Exerted by Preparation for Periodontal Tissue>>

<Subject 1> (Example)

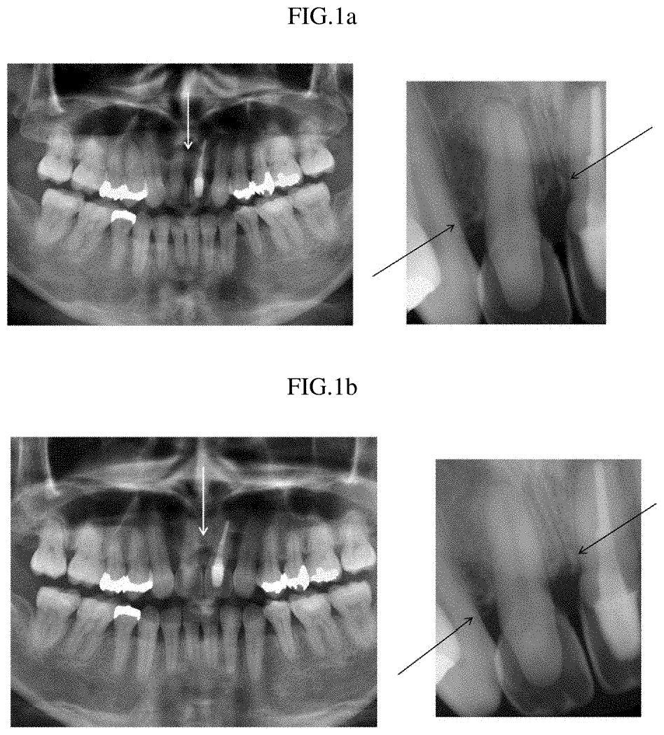

FIG. 1a is panoramic X-ray photographs of a 47-year-old woman with alveolar bone defects caused by periodontal disease and bruxism. The amount of maxillary anterior tooth alveolar bone of this woman was reduced, and slight mobility (M1) was also observed. In FIG. 1a, a bone loss occurred and a black-looking area was increased. This indicates no alveolar crest absorption, resulting in a black spot. Alveolar bone lines are also obscure due to the bone loss.

To the maxillary anterior tooth bone of the patient, 2 ml of the preparation in Example 1 was injected and administered 6 times at a frequency of once a month.

FIG. 1b is photographs after the sixth injection. The alveolar bone of the maxillary anterior tooth increased, and the slight mobility (M1) before surgery was also improved when compared to those before the treatment. In addition, the bone density is increased, the black-looking area is reduced, and the white area due to the formation of a new bone is increased. The missing bone at the alveolar crest is found to be newly formed, and the area appears white. This patient suffered from gingival recession, but after the sixth injection, the gingival recession was recovered to some extent.

<Subject 2> (Example)

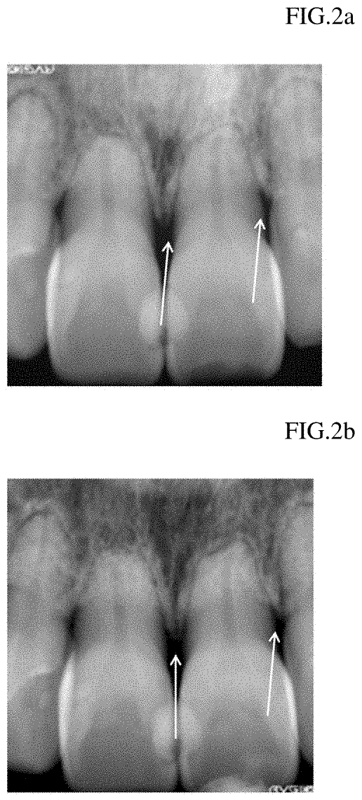

FIG. 2a is a panoramic X-ray alveolar bone photograph of a 52-year-old woman who had a strong hit on the maxillary anterior tooth part and had slight mobility (grade II) and palpation pain in the right and left anterior teeth.

To the alveolar bone and the periodontal ligament of the patient, 2 ml of the preparation in Example 1 was injected and administered 4 times at a frequency of once a month.

FIG. 2b is a photograph after the fourth injection. It can be seen that the bone density of the maxillary anterior tooth increases and the position of the alveolar crest becomes higher. In addition, it can be seen that the white bone area of the adjacent portion also increased, and the bone was thickened. The mobility and percussion pain disappeared after the second injection.

<Subject 3> (Example)

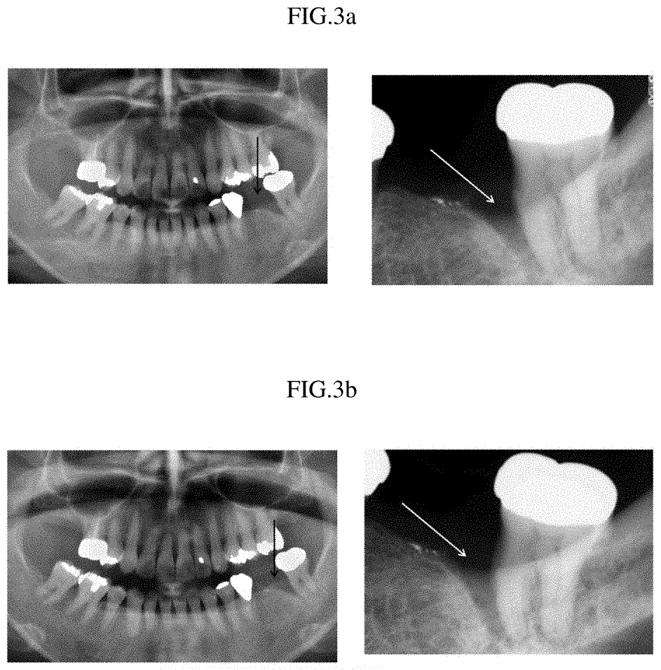

FIG. 3a is panoramic X-ray alveolar bone photographs of a 47-year-old woman with bone resorption and loss observed at the mandibular left molar (mandibular left at tooth position 7) on the mesial side due to periodontal disease deterioration caused by smoking. In this photograph, the bone on the mesial side is reduced and appears black, and the alveolar bone line is also obscure due to the bone loss.

To the mandibular left molar bone of the patient, 2 ml of the preparation in Example 1 was injected and administered 6 times at a frequency of once a month.

FIG. 3b is photographs after the fourth injection. A new bone starts to be formed, the white area increases, and an alveolar bone line is formed.

<Subject 4> (Example)

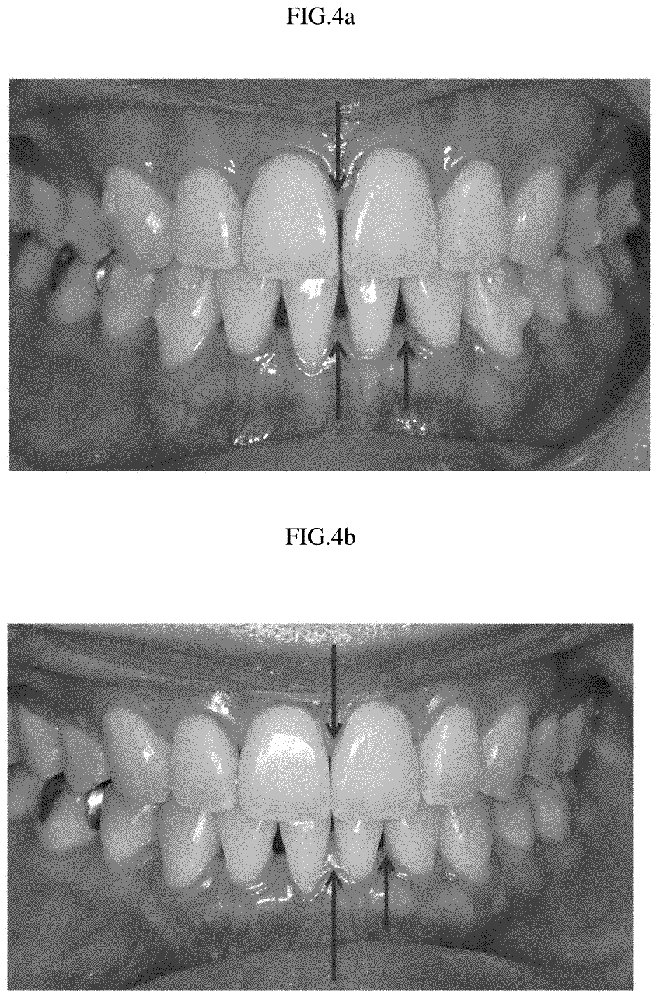

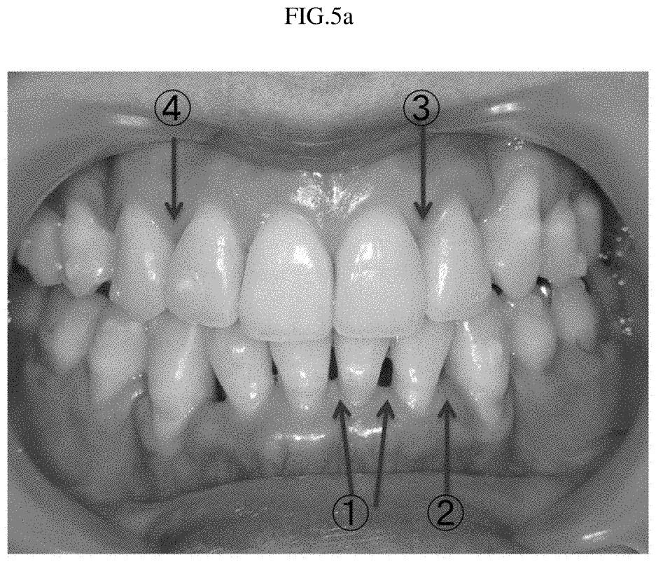

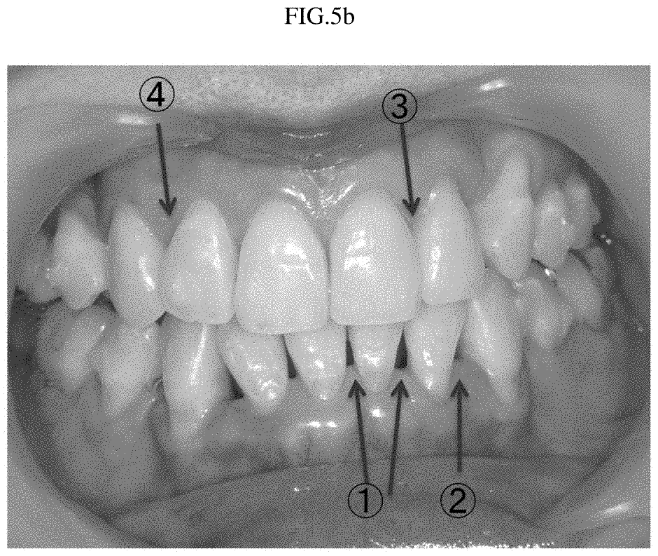

FIG. 4a is a photograph of a periodontal tissue of a 26-year-old woman with gingival recession caused by periodontal disease and bruxism. Gingival recession is observed in the mandibular anterior tooth part and the maxillary anterior tooth parts. There is a large interdental space between the maxillary anterior teeth, and large black triangles also occur between the mandibular anterior teeth. The patient herself was particularly aesthetically concerned.

To the gums of the mandibular and maxillary anterior teeth of the patient, 2 ml of the preparation in Example 2 was injected and administered 6 times at a frequency of about once a month.

FIG. 4b is a photograph after the sixth injection. In the mandibular anterior tooth parts, the area of gum attached increased and their papilla was thickened. As a result, the interdental space was decreased, and the black triangles became inconspicuous. In addition, the gum of the interdental papilla between the mandibular center teeth at tooth position 1 is found to be markedly increased. Furthermore, it can be seen that the gum of the maxillary anterior tooth papilla was increased and thickened, so that the interdental space became inconspicuous. The patient herself was also aware of the effect and was very satisfied.

<Subject 5> (Example)

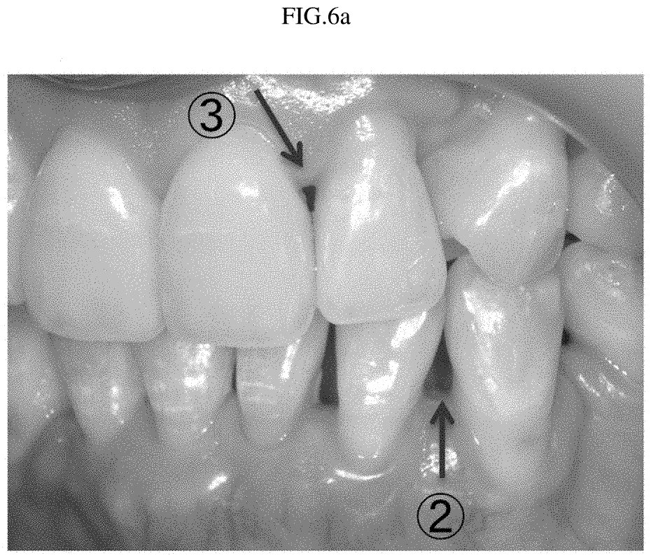

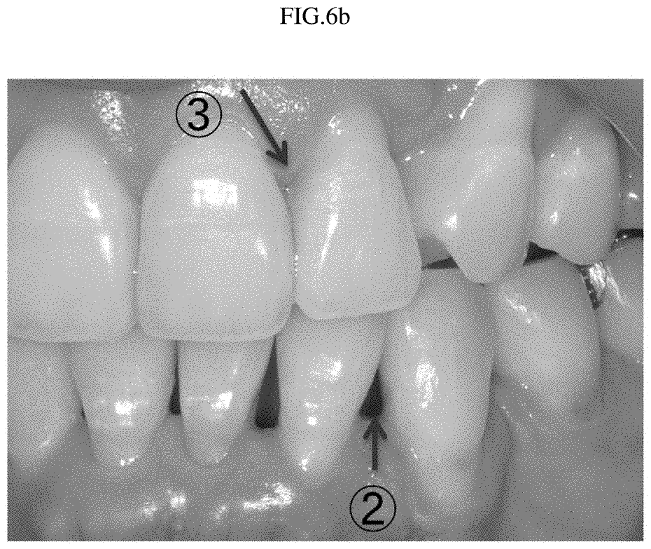

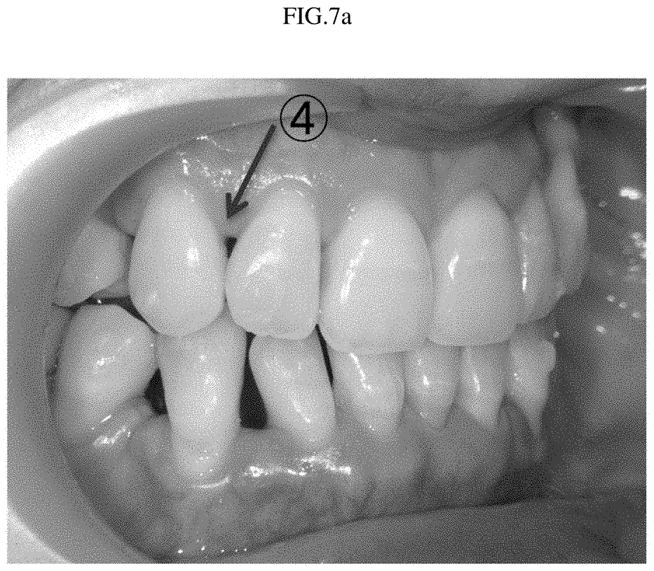

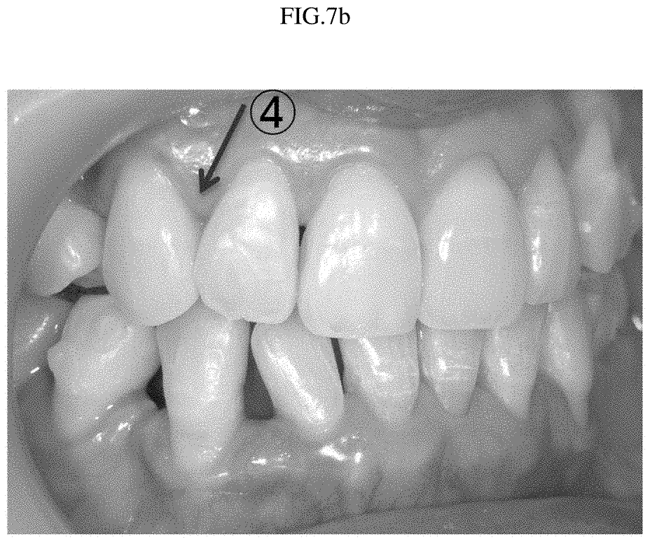

FIGS. 5a, 6a, and 7a are front, right, and left photographic views, respectively, of a periodontal tissue of a 47-year-old woman with gingival recession caused by periodontal disease and malocclusion. In particular, large black triangles occurred in the mandibular anterior tooth parts, and the patient herself was particularly aesthetically concerned.

To the gums of the mandibular and maxillary anterior teeth of the patient, 2 ml of the preparation in Example 2 was injected and administered 5 times at a frequency of about once a month.

FIGS. 5b, 6b, and 7b are front, right, and left photographic views, respectively, of the periodontal tissue after the fifth injection. In the mandibular anterior tooth parts of “1” and “2” in the photograph, the area of gum attached increased and the papilla was also thickened, so that their interdental space decreased and the black triangles became inconspicuous. It can be seen that the gum of the maxillary anterior tooth papilla of any of “3” or “4” in the photograph increases and is thickened, so that the black triangles disappear. The patient herself was also aware of the effect and was very satisfied.

<Subject 6> (Reference Example)

To a 26-year-old woman with gingival recession after orthodontics, 1 ml of hyaluronic acid (Restylane VITAL SKINBOOSTERS (registered trademark)) was injected using a routine procedure 3 times at a frequency of once every 3 to 4 weeks. Although strong pain occurred at the time of treatment, gingiva was regenerated to some extent.

<Subject 7> (Reference Example)

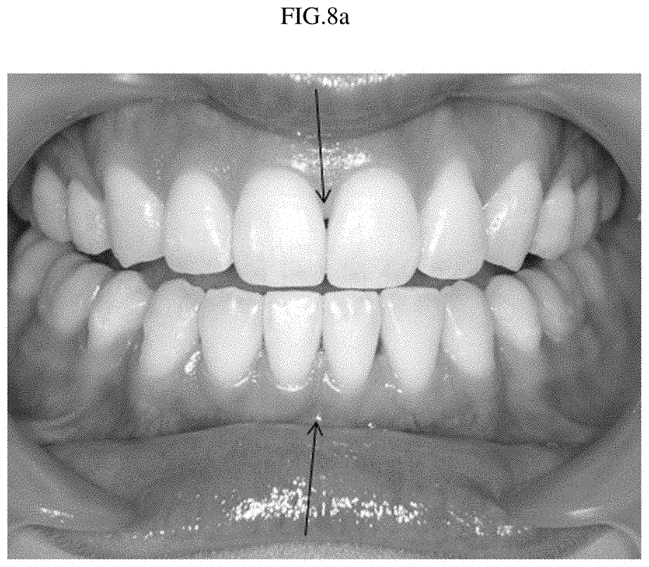

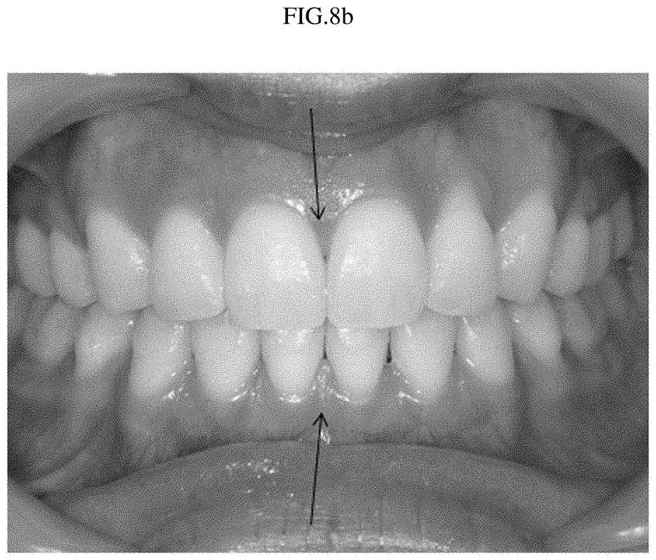

FIG. 8a is a photograph of a periodontal tissue of a 29-year-old woman with gingival recession after orthodontics. Gingival recession is observed in the mandibular anterior tooth part and the maxillary anterior tooth parts. There is an interdental space between the maxillary anterior teeth, so that a black triangle occurs.

To the gums of the mandibular and maxillary anterior teeth of the patient, 1 ml of the preparation containing only the above-described culture supernatant was injected and administered 3 times at a frequency of once every 3 to 4 weeks.

FIG. 8b is a photograph of the periodontal tissue after the third injection. It can be clearly seen that when compared to before the treatment, the gum of the maxillary anterior tooth part is increased and thickened, so that the black triangle becomes inconspicuous. In the mandibular anterior tooth part, the area of gum increased and their papilla was thickened.

<Subject 8> (Reference Example)

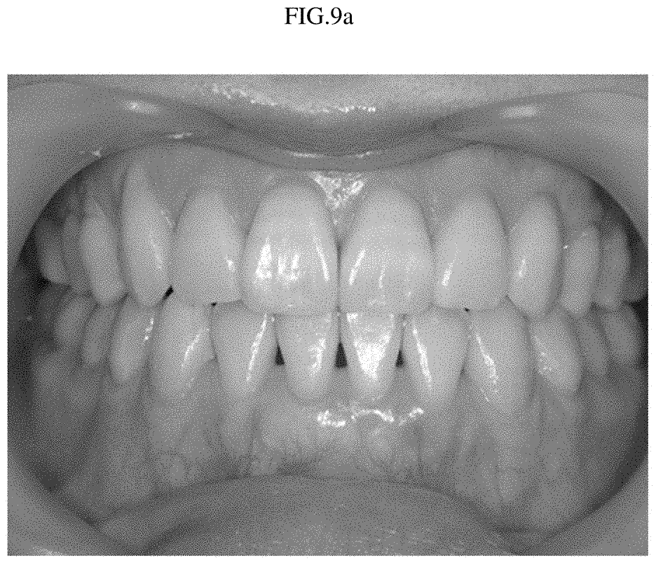

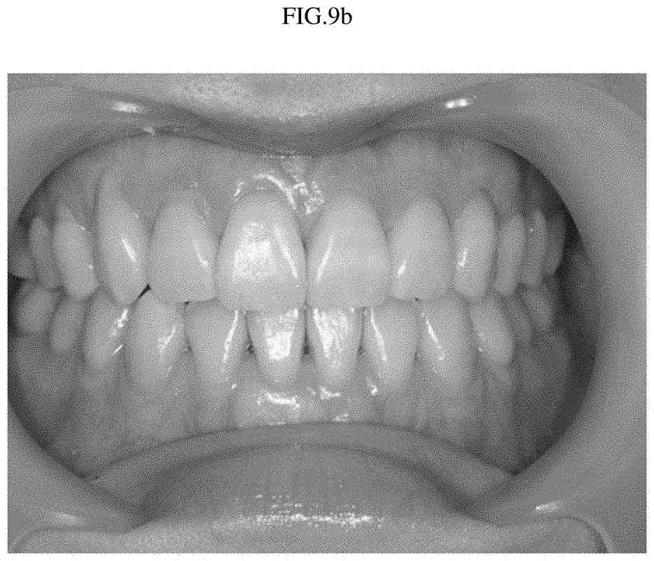

FIG. 9a is a photograph of a periodontal tissue of a 28-year-old woman with periodontal disease and gingival recession after orthodontics. Gingival recession and a decrease in attached gum are observed in the mandibular and maxillary anterior tooth parts, and their gums are thin. In the mandibular anterior tooth parts, gingival recession causes a loss of interdental papilla, so that black triangles occur.

To the gums of the mandibular and maxillary anterior teeth of the patient, 1 ml of the preparation containing only the above-described culture supernatant was injected and administered 9 times at a frequency of once every 3 to 4 weeks.

FIG. 9b is a photograph of the periodontal tissue after the ninth injection. It can be seen that when compared to before the treatment, the gums of the maxillary and mandibular anterior teeth are increased and thickened. In the mandibular anterior tooth parts, in particular, it can be clearly seen that the area of gum attached increased and the papilla was also thickened, so that their interdental space decreased substantially and the black triangles became inconspicuous. Also, in the mandibular anterior tooth parts, the area of gum increased and the papilla was also thickened, so that the black triangles became small.

<Subject 9> (Reference Example)

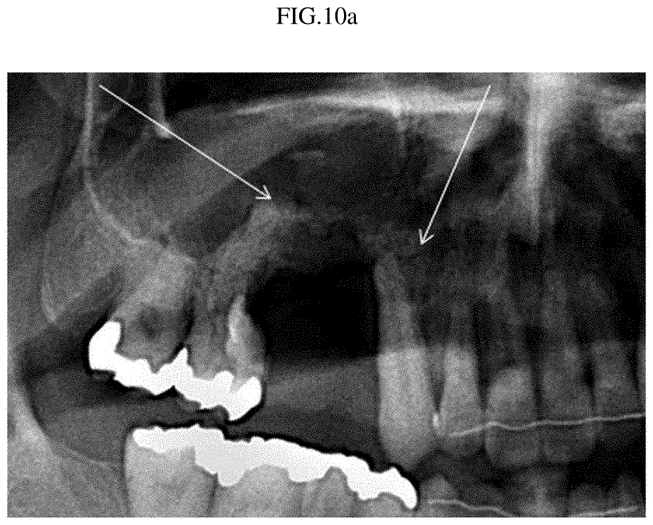

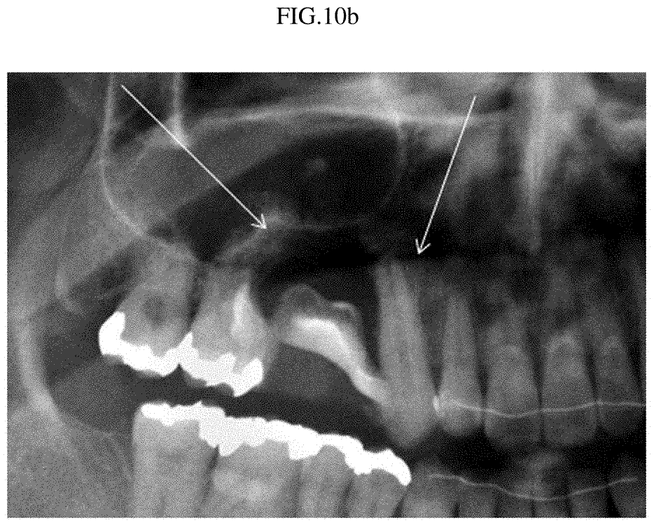

FIG. 10a is a panoramic X-ray image of a periodontal tissue of a 35-year-old man with alveolar bone defects due to severe periodontal disease. At the time of initial diagnosis, severe periodontal disease caused marked unevenness at the missing tooth portion, and the periapical bone of a canine was also thin.

To the alveolar bone of the patient, 1 ml of the preparation containing only the above-described culture supernatant was injected and administered 4 times at a frequency of once every 3 to 4 weeks.

FIG. 10b is a panoramic X-ray photograph of the periodontal tissue after the fourth injection. It can be seen that when compared to before the treatment, the alveolar bone is increased and the bone is regenerated in the missing tooth portion. It can be also seen that the bone is regenerated in the periapical tooth part of the canine, and the white-looking area is increased. However, it cannot be said that the alveolar bone regeneration potential is as high as in the case of the autologous conditioned serum-containing preparation.

<Subject 10> (Reference Example)

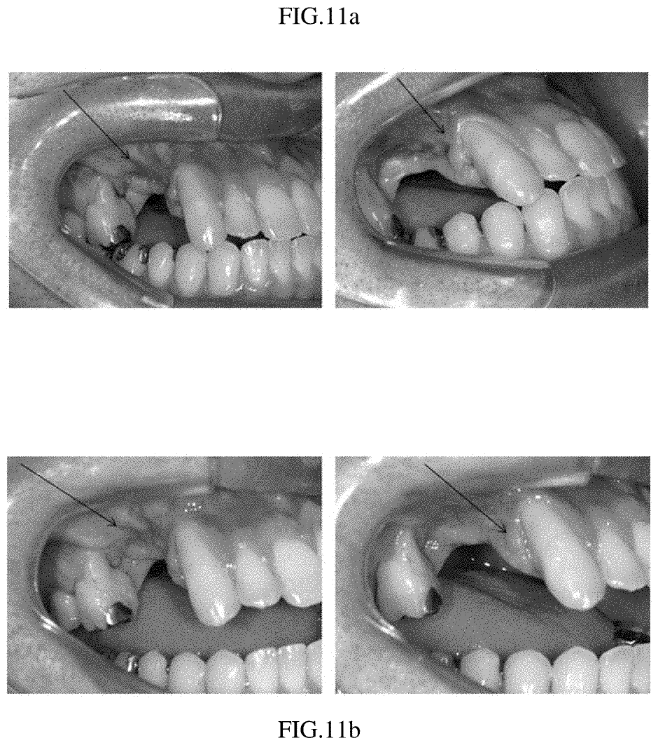

FIG. 11a is photographs of a periodontal tissue of a man with alveolar bone defects due to severe periodontal disease. At the time of initial diagnosis, severe periodontal disease caused a loss of the gum of the missing tooth portion and a loss of the gum on the distal side of the canine. This resulted in mobility when the canine was touched.

To the alveolar bone of the patient, 1 ml of the preparation containing only the above-described culture supernatant was injected and administered 3 times at a frequency of once every 3 to 4 weeks.

FIG. 11b is photographs of the periodontal tissue after the third injection. Compared to before the treatment, the gum of the missing tooth portion was regenerated, and the gum on the distal side of the canine was thickened. As a result, the mobility of the canine was treated substantially. However, it cannot be said that the alveolar bone regeneration potential is as high as in the case of the autologous conditioned serum-containing preparation.

<Conclusion>

The respective patients had individual differences. Nevertheless, in summary, Table 1 below can collectively provide the effects of regeneration of the periodontal tissue and the patient's satisfaction in the case of using the conventional hyaluronic acid preparation, the culture supernatant-containing preparation, or the preparation of the present invention. The effect on the regeneration potential was judged by a dentist(s). Here, “0” means no effect, “1” means that there is an effect or means slightly satisfactory, “2” means effective or satisfactory, and “3” means very effective or very satisfactory.

TABLE 1

| | Alveolar | | |
|---|---|---|---|
| | bone | Gingival | Patient's |
| | regeneration | regeneration | satisfaction |
| Hyaluronic acid | 0 | 1 | 1 |
| Culture supernatant | 1 | 2 | 2 |
| Autologous conditioned serum | 2 | 2 | 2 |
| Antologous conditioned serum | 2 | 3 | 3 |
| and Culture supernatant | | | |

That is, use of the autologous conditioned serum-containing preparation made it possible to very effectively regenerate an alveolar bone, which had been impossible using the conventional technology. In addition, gingival regeneration was effectively achieved from the beginning of the treatment. In particular, it has been found that a preparation containing autologous conditioned serum and a culture supernatant has very high periodontal tissue regeneration performance.

<<Pharmacological Effects of Kit Containing Preparation of Example and Hyaluronic Acid Preparation>>

To a plurality of patients with gingival recession and/or alveolar bone defects, the preparation of Example 1 or 2 was injected and administered multiple times at a frequency of once every 3 to 4 weeks. Thereafter, a hyaluronic acid-containing preparation was injected into the gum around the alveolar bone.

As a result, a high periodontal tissue regeneration effect as in Examples 1 and 2 was obtained. Also, the black triangles at the gums and the tooth mobility were almost completely eliminated. All the patients were thus very satisfied.

The preparation of Example 1 or 2 was additionally injected and administered multiple times at the site where the hyaluronic acid was injected. This has been found to elicit a higher periodontal tissue regeneration effect. This seems to be because administration of hyaluronic acid causes swelling of gum to impart stretch stimulation to cells of the gum, thereby enhancing fibroblast activity (e.g., collagen synthesis potential, proliferation capability) to promote gingival regeneration. In addition, it is considered that the tissue regeneration potential of the preparation of Example 1 or 2 was effectively exhibited around the tissue stretched by hyaluronic acid, and a synergistic effect was thus exerted.

1. A method for producing an injectable preparation for a periodontal tissue, comprising autologous conditioned serum and a culture supernatant,
wherein the autologous conditioned serum is obtained by a method comprising the steps of:
incubating blood collected from a patient using the preparation for a periodontal tissue with bringing the blood into contact with glass in a range of 30° C. to 45° C. for 1 hour or longer; and
separating serum from the incubated blood, and
wherein the culture supernatant is a culture supernatant of adipose tissue-derived mesenchymal stromal cells, epidermis-derived epithelial cells, or dental pulp-derived mesenchymal stem cells.

2. (canceled)

3. The method for producing the injectable preparation for a periodontal tissue according to claim 1, wherein the culture supernatant is obtained by a method comprising:
a first culturing step of culturing cells by using a first medium;
a second culturing step of culturing, after the first culturing step, the cells by using, as a culture medium, a second medium different from the first medium; and
a step of obtaining, after the second culturing step, a culture supernatant containing the second medium.

4. The method for producing the injectable preparation for a periodontal tissue according to claim 3, wherein the second culturing step is performed in a non-CO₂ atmosphere.

5. The method for producing the injectable preparation for a periodontal tissue according to claim 4, wherein the second medium comprises a calcium ion and a buffering agent.

6. The preparation for a periodontal tissue according to claim 5, wherein the buffering agent is selected from a Good's buffer.

7. The preparation for a periodontal tissue according to claim 6, wherein the Good's buffer is HEPES.

8. A method for producing kit for a periodontal tissue, comprising combining the injectable preparation for a periodontal tissue obtained by the method according to claim 1 with a hyaluronic acid-containing injectable preparation for a periodontal tissue.

9. (canceled)

10. (canceled)

11. (canceled)

12. A method for treatment of a periodontal tissue of human or non-human mammal by using the injectable preparation produced by the method according to claim 1.

13. A method for treatment of a periodontal tissue of human or non-human mammal by using the kit produced by the method according to claim 7.
